(19) United States
(12) Patent Application Publication
(10) Pub. No.: US 2025/0170389 A1
(43) Pub. Date: May 29, 2025

(54) ADAPTIVE NEUROMODULATION IN RESPONSE TO LOSS OF SYSTEM INTEGRITY

(71) Applicant: Boston Scientific Neuromodulation Corporation, Valencia, CA (US)

(72) Inventors: Mahsa Malekmohammadi, Sherman Oaks, CA (US); Lisa Denise Moore, Glendale, CA (US); Richard Mustakos, Simi Valley, CA (US); Peter J. Yoo, Burbank, CA (US); Rajat Shamachar Shivacharan, Germantown, MD (US)

(73) Assignee: Boston Scientific Neuromodulation Corporation, Valencia, CA (US)

(21) Appl. No.: 18/956,972

(22) Filed: Nov. 22, 2024

Related U.S. Application Data
(60) Provisional application No. 63/603,478, filed on Nov. 28, 2023.

(51) Int. Cl. A61N 1/02; A61N 1/36; A61N 1/372

(52) U.S. Cl. CPC A61N 1/025; A61N 1/36132; A61N 1/36139; A61N 1/36185; A61N 1/37247

(57) ABSTRACT

Methods and systems for adapting a neuromodulation system to changes in system integrity. A system may check system integrity in response to the passage of time or to an asynchronous trigger. If a loss of system integrity is identified, changes to therapy program parameters are determined and tested for satisfactory performance. The changes to a therapy program may include identifying a new therapeutic target. Physician approval of the change process, as well as final program parameters, is contemplated.

### CROSS-REFERENCE TO RELATED APPLICATIONS

This application claims the benefit of U.S. Provisional Patent Application Ser. No. 63/603,478, filed Nov. 28, 2023, which is incorporated herein by reference.

### BACKGROUND

Neuromodulation therapies have been shown to provide numerous benefits for patients. Various such systems are known, including, for example, systems for spinal cord stimulation (SCS), deep brain stimulation (DBS), Vagus nerve stimulation (VNS), Sacral nerve stimulation (SNS) and/or peripheral nerve stimulation (PNS). Therapy output by such systems is carefully planned and calibrated to a given patient's needs as well as system designs, including implant position, disease state, etc. However, such systems are susceptible to a loss of system integrity.

Loss of system integrity may include damage to various components, including leads and implantable pulse generators for example. Devices can be damaged or dislodged due to a patient falling, for example, or may degrade over a longer period of time. System integrity may be lost suddenly or may occur due to gradual changes in the system and/or components. Typically, a loss of system integrity is determined by comparing one or more measurable parameters, such as lead impedance, to a threshold; once the threshold is crossed, alerts may be issued to a physician and/or patient, and the system often ceases to deliver therapy until clinical follow up takes place.

New and alternative systems and methods are desired to allow compensatory action to be taken by a neuromodulation system in response to sudden or gradual loss of system integrity.

### Overview

The present inventors have recognized, among other things, that a problem to be solved is the need to allow compensatory action to be taken by a neuromodulation system in response to sudden or gradual loss of system integrity.

A first illustrative and non-limiting example takes the form of a neuromodulation system comprising an implantable pulse generator including a housing containing operational circuitry having therein a microcontroller and a memory, the memory storing patient data and readable instructions for one or more programs for treating the patient; wherein the operational circuitry is configured to: apply a first set of program parameters to deliver therapy to a patient, the first set of program parameters stored in the memory; check system integrity; determine a loss of system integrity; obtain or determine a second set of program parameters in response to the loss of system integrity; apply the second set of program parameters to deliver therapy to the patient; obtain patient feedback in response to the application of the second set of program parameters to deliver therapy to the patient; and determine whether the patient feedback indicates acceptability of the second set of program parameters.

Additionally or alternatively, the system includes a patient remote control, wherein the operational circuitry comprises a communications circuit adapted to communicate with the patient remote control, wherein: the operational circuitry is configured to check system integrity by obtaining, from the patient remote control, data indicating whether the patient is satisfied with therapy delivered using the first set of program parameters; and the operational circuitry determines a loss of system integrity in response to the data from the patient remote control indicating: the patient was previously satisfied with therapy delivered using the first set of program parameters; and that the patient is no longer satisfied with therapy delivered using the first set of program parameters.

Additionally or alternatively, the system includes a patient remote control and a lead coupled to the implantable pulse generator, wherein the operational circuitry comprises a communications circuit adapted to communicate with the patient remote control, wherein: the operational circuitry is configured to check system integrity by obtaining one or more of position or impedance data related to the lead, and obtaining, from the patient remote control, data indicating whether the patient is satisfied with therapy delivered using the first set of program parameters; and the operational circuitry determines a loss of system integrity in response to finding that each: a) the position or impedance data related to the lead has changed from a prior state to a current state; and b) the data from the patient remote control indicates: the patient was previously satisfied with therapy delivered using the first set of program parameters; and the patient is no longer satisfied with therapy delivered using the first set of program parameters.

Additionally or alternatively, the system includes a lead having a proximal end for coupling to the pulse generator and a distal end with a plurality of electrodes thereon, wherein: the first therapy program is delivered using a first electrode of the plurality of electrodes on the lead; the operational circuitry is configured to measure impedance at the first electrode while therapy is delivered using the first electrode; and the operational circuitry determines a loss of system integrity in response to finding that the measured impedance has crossed above a first threshold or below a second threshold, indicating a potential failure with the first electrode.

Additionally or alternatively, the memory contains a clinical effects map indicating beneficial combinations of therapy amplitude and lead position that cause clinical benefits and side-effect combinations of therapy amplitude and lead position that cause side effects in the patient; the first therapy program uses a first combination of therapy amplitude and lead position that cause clinical benefits having a first location on the clinical effects map; and the operational circuitry is configured to determine a second set of program parameters in response to the loss of system integrity by identifying a second location on the clinical effects map that uses a combination of therapy amplitude and lead position which the clinical effects map indicates will cause clinical benefits and not side effects.

Additionally or alternatively, the memory contains an anatomical mapping of target locations and avoid locations, relative to the lead in the patient; the first therapy program is configured to issue therapy to a first target location; and the operational circuitry is configured to determine the second set of program parameters in response to the loss of system integrity by identifying a second target location that is not the first target location, and configuring the second set of program parameters to issue therapy to the second target location.

Additionally or alternatively, the system includes a patient remote control having a remote control memory storing a clinical effects map indicating benefit combinations of therapy amplitude and lead position that cause clinical benefits and side-effect combinations of therapy amplitude and lead position that cause side effects in the patient; the first therapy program uses a first combination of therapy amplitude and lead position that cause clinical benefits having a first location on the clinical effects map; the remote control is configured to identify a second location on the clinical effects map that uses a second combination of therapy amplitude and lead position which the clinical effects map indicates will cause clinical benefits and not side effects; the remote control is configured to determine the second set of program parameters from the second combination of therapy amplitude and lead position; and the operational circuitry is configured to obtain the second set of program parameters from the patient remote control.

Additionally or alternatively, the system includes a patient remote control having a remote control memory storing an anatomical mapping of target locations and avoid locations, relative to the lead in the patient; the first therapy program is configured to issue therapy to a first target location; and the remote control is configured to determine the second set of program parameters in response to the loss of system integrity by identifying a second target location that is not the first target location, and configuring the second set of program parameters to issue therapy to the second target location; and the operational circuitry is configured to obtain the second set of program parameters from the patient remote control.

Additionally or alternatively, the memory contains a plurality of sets of program parameters including each of the first set of program parameters and the second set of program parameters; the operational circuitry stores an active list of which of the plurality of sets of program parameters are available for use at any given time; when the operational circuitry determines the loss of system integrity, the first set of program parameters is in use, and the second set of program parameters is not in use; the operational circuitry is configured to remove the first set of program parameters from the active list, add the second set of program parameters to the active list, and obtain the second set of program parameters from the memory in response to determining the loss of system integrity. Additionally or alternatively, the second set of program parameters does not use the first electrode in therapy delivery.

Additionally or alternatively, the operational circuitry is further configured to: generate an alert to request a change in response to the loss of system integrity; and receive a response approving the change; wherein the operational circuitry is configured to wait to apply the second set of program parameters to deliver therapy to the patient until after receiving the response approving the change.

Additionally or alternatively, the operational circuitry is configured to send the second set of program parameters with the request. Additionally or alternatively, the alert is issued to a physician.

Additionally or alternatively, the system includes a patient remote control configured to present queries to a patient and receive responses therefrom, wherein the operational circuitry is configured to obtain patient feedback in response to the application of the second set of program parameters to deliver therapy to the patient by communicating to the patient remote control to present a query to the patient, and to then receive an indication of the patient feedback from the patient remote control.

Additionally or alternatively, the system includes a motion sensor, wherein the operational circuitry is configured to obtain patient feedback in response to the application of the second set of program parameters to deliver therapy to the patient by obtaining data from the motion sensor to analyze one or more of gait or tremor.

Another illustrative and non-limiting example takes the form of a method of operation in a neuromodulation system having an implantable pulse generator including a housing containing operational circuitry configured to deliver therapy to the patient using one or more therapy programs, the method comprising: applying a first set of program parameters to deliver therapy to a patient, the first set of program parameters being in a first therapy program; checking system integrity; determining a loss of system integrity; in response to determining a loss of system integrity, applying a second set of program parameters to deliver therapy to the patient, the second set of program parameters being in a second therapy program; obtaining patient feedback in response to the application of the second set of program parameters to deliver therapy to the patient; and determining whether the patient feedback indicates acceptability of the second set of program parameters.

Additionally or alternatively, the system includes a patient remote control in communication with the implantable pulse generator, wherein: checking system integrity includes obtaining, from the patient remote control, data indicating whether the patient is satisfied with therapy delivered using the first set of program parameters; and determining a loss of system integrity includes finding that: the patient was previously satisfied with therapy delivered using the first set of program parameters; and that the patient is no longer satisfied with therapy delivered using the first set of program parameters.

Additionally or alternatively, the system includes a patient remote control in communication with the implantable pulse generator, and a lead coupled to the implantable pulse generator, wherein: checking system integrity includes obtaining one or more of position or impedance data related to the lead, and obtaining, from the patient remote control, data indicating whether the patient is satisfied with therapy delivered using the first set of program parameters; and determining a loss of system integrity includes finding each of: the position or impedance data related to the lead has changed from a prior state to a current state; and the data from the patient remote control indicates: the patient was previously satisfied with therapy delivered using the first set of program parameters; and the patient is no longer satisfied with therapy delivered using the first set of program parameters.

Additionally or alternatively, the system includes a lead having a proximal end for coupling to the pulse generator and a distal end with a plurality of electrodes thereon, wherein the first therapy program is delivered using a first electrode of the plurality of electrodes on the lead; the method comprises measuring impedance at the first electrode while therapy is delivered using the first electrode; and determining a loss of system integrity is performed by finding the measured impedance has crossed above a first threshold or below a second threshold, indicating a potential failure with the first electrode.

Additionally or alternatively, the operational circuitry includes a memory storing instructions for executing the one or more therapy programs; the memory contains a clinical effects map indicating beneficial combinations of therapy amplitude and lead position that cause clinical benefits and side-effect combinations of therapy amplitude and lead position that cause side effects in the patient; the first therapy program uses a first combination of therapy amplitude and lead position that cause clinical benefits having a first location on the clinical effects map; and the method comprises determining the second set of program parameters in response to the loss of system integrity by identifying a second location on the clinical effects map that uses a combination of therapy amplitude and lead position which the clinical effects map indicates will cause clinical benefits and not side effects.

Additionally or alternatively, the operational circuitry includes a memory storing instructions for executing the one or more therapy programs; the memory contains an anatomical mapping of target locations and avoid locations, relative to the lead in the patient; the first therapy program is configured to issue therapy to a first target location; and the method comprises determining the second set of program parameters in response to the loss of system integrity by identifying a second target location that is not the first target location, and configuring the second set of program parameters to issue therapy to the second target location.

Additionally or alternatively, the neuromodulation system includes a patient remote control having a memory storing a clinical effects map indicating benefit combinations of therapy amplitude and lead position that cause clinical benefits and side-effect combinations of therapy amplitude and lead position that cause side effects in the patient; the first therapy program uses a first combination of therapy amplitude and lead position that cause clinical benefits having a first location on the clinical effects map; the method includes: identifying a second location on the clinical effects map that uses a second combination of therapy amplitude and lead position which the clinical effects map indicates will cause clinical benefits and not side effects; determining the second set of program parameters from the second combination of therapy amplitude and lead position; and the patient remote control communicating the second set of program parameters to the implantable pulse generator.

Additionally or alternatively, the neuromodulation system includes a patient remote control having a memory storing an anatomical mapping of target locations and avoid locations, relative to the lead in the patient; the first therapy program is configured to issue therapy to a first target location; and the method includes: determining the second set of program parameters in response to the loss of system integrity by identifying a second target location that is not the first target location, and configuring the second set of program parameters to issue therapy to the second target location; and the patient remote control communicating the second set of program parameters to the implantable pulse generator.

Additionally or alternatively, the operational circuitry includes a memory storing instructions for executing at least the first set of program parameters and the second set of program parameters, and an active list identifying which of the plurality of sets of program parameters are available to be selected by a patient for use at any given time; the method includes determining that the second set of program parameters is not in-use at the time the loss of system integrity occurs, and the first set of program parameters is in use at the time the loss of system integrity occurs; and the method includes removing the first set of program parameters from the active list, and adding the second set of program parameters to the active list.

Additionally or alternatively, the second set of program parameters does not use the first electrode in therapy delivery.

Additionally or alternatively, the method further includes generating an alert to request a change in response to the loss of system integrity; and receiving a response to the alert, approving the change; wherein the method includes waiting to apply the second set of program parameters to deliver therapy to the patient until after receiving the response approving the change. Additionally or alternatively, the step of generating an alert to request a change includes sending the second set of program parameters with the request. Additionally or alternatively, the step of generating an alert to request a change includes alerting a physician.

Additionally or alternatively, the system also includes a patient remote control configured to present queries to a patient and receive responses therefrom, wherein the step of obtaining patient feedback includes communicating to the patient remote control to present a query to the patient, and then receiving an indication of the patient feedback from the patient remote control.

Additionally or alternatively, the step of obtaining patient feedback includes monitoring a patient motion sensor to analyze one or more of gait or tremor. Additionally or alternatively, the patient motion sensor is contained in or on the implantable pulse generator. Additionally or alternatively, the patient motion sensor is a component of a wearable device, and the implantable pulse generator contains communication circuitry configured to communicate with the wearable device.

Additionally or alternatively, the system also includes a patient remote control adapted to communicate with the implantable pulse generator and having an interface for communicating with the patient; the patient motion sensor is a component of a wearable device; and the patient remote control is further adapted to communicate with the wearable device.

This overview is intended to provide an introduction to the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows a deep brain stimulation (DBS) system;

FIG. 2 shows an illustrative implantable pulse generator (IPG);

FIG. 3 shows a spinal cord stimulation (SCS) system;

FIG. 4 shows an illustrative method of adapting to loss of system integrity;

FIG. 5 shows an illustrative method of selecting a new a therapy program;

FIGS. 6A-6C show the use of a clinical effects map to configure a new therapy program; and

FIGS. 7A-7C show the use of anatomical mapping to configure a new therapy program.

### DETAILED DESCRIPTION

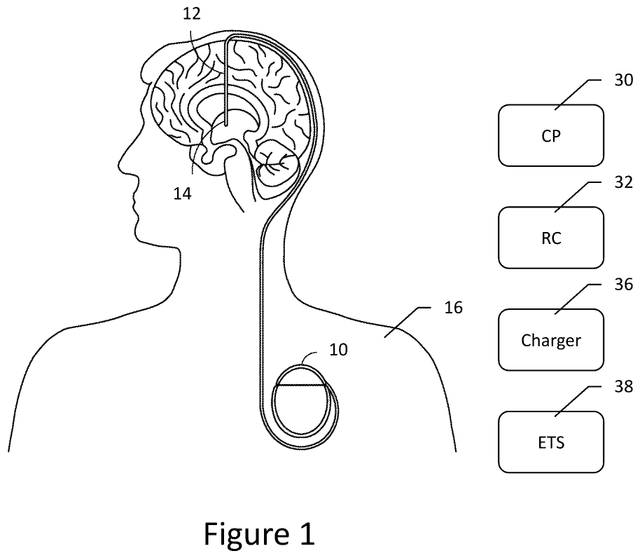

FIG. 1 shows an illustrative DBS system implanted in a patient. The system comprises an implantable pulse generator (IPG) 10, shown implanted in the pectoral region of a patient 16. The IPG 10 is coupled to a lead 12 which extends subcutaneously to the head of the patient 16, through a burr hole formed in the patient's skull, and then into the brain. In the example shown, the lead 12 includes a plurality of electrodes positioned near the distal end 14 of the lead. The lead 12 may be placed at any suitable location of the brain where a target for therapy is identified. For example, a lead 12 may be positioned so that the distal end 14 is near the mid-brain and/or various structures therein that are known in the art for use in providing stimulation to treat various diseases.

DBS may be targeted, for example, and without limitation, at neuronal tissue in the thalamus, the globus pallidus, the subthalamic nucleus, the pedunculopontine nucleus, substantia nigra pars reticulate, the cortex, the globus pallidus externus, the medial forebrain bundle, the periaquaductal gray, the periventricular gray, the habenula, the subgenual cingulate, the ventral intermediate nucleus, the anterior nucleus, other nuclei of the thalamus, the zona incerta, the ventral capsule, the ventral striatum, the nucleus accumbens, and/or white matter tracts connecting these and other structures. Data related to DBS may include the identification of neural tissue regions determined analytically to relate to side effects or benefits observed in practice. “Targets” as used herein are brain structures associated with therapeutic benefits, in contrast to avoidance regions or “Avoid” regions which are brain structures associated with side effects.

Conditions to be treated may include dementia, Alzheimer's disease, Parkinson's disease, dyskinesias, tremors, depression, anxiety or other mood disorders, sleep related conditions, etc. Therapeutic benefits may include, for example, and without limitation, improved cognition, alertness, and/or memory, enhanced mood or sleep, elimination, avoidance or reduction of pain or tremor, reduction in motor impairments, and/or preservation of existing function and/or cellular structures, such as preventing loss of tissue and/or cell death. Therapeutic benefits may be monitored using, for example, patient surveys, performance tests, and/or physical monitoring such as monitoring gait, tremor, etc. Side effects can include a wide range of issues such as, for example, and without limitation, reduced cognition, neuroinflammation, alertness, and/or memory, degraded sleep, depression, anxiety, unexplained weight gain/loss, tinnitus, pain, tremor, etc. These are just examples, and the discussion of ailments, benefits and side effects is merely illustrative and not exhaustive.

The illustrative system of claim 1 includes various external devices. A clinician programmer (CP) 30 may be used to determine/select therapy programs, including steering (further explained below) as well as stimulation parameters. The CP 30 can be used by a physician, or at the direction of a physician, to obtain data from and provide instructions the IPG 10 via suitable communications protocols such as Bluetooth or MedRadio or other wireless communications standards, and/or via other modalities such as inductive telemetry. Stimulation parameters may include amplitude of stimulation pulses, frequency or repetition rate of stimulation pulses, pulse width of stimulation pulses, and more complex parameters such as burst definition, as are known in the art. Biphasic square waves are commonly used, though nothing in the present invention is limited to biphasic square waves, and ramped, triangular, sinusoidal, monophasic and other stimulation types may be used as desired.

The CP 30 may be, for example and without limitation, a computer such as a laptop or tablet computer. The CP 30 therefore includes a microcontroller and/or microprocessor, and associated memory. The memory may take any suitable form (RAM, ROM, Flash, etc.), and stores machine readable instructions allowing the processor to perform the methods disclosed herein. To the extent Bluetooth is used as a communications protocol, the RF circuitry may be included in the device as a communications circuitry, located internal to the CP 30. If some other communications technology (inductive or Medradio) is used, or if range is limited by the IPG for example, the communications circuit may be provided via a wand having specialized circuitry (for Bluetooth, Medradio, or inductive telemetry) therein that couples, for example, to a USB port on the CP 30. The CP 30 may include a user interface, such as a screen or touchscreen, keyboard, mouse, trackball, etc. allowing the user to provide instructions and make choices.

A patient remote control (RC) 32 can be used by the patient to perform various actions relative to the IPG 10. These may be physician defined options, and may include, for example, turning therapy on and/or off, entering requested information (such as answering questions about activities, therapy benefits and side effects), and making (limited) adjustments to therapy such as selecting from available therapy programs and adjusting, for example, amplitude settings. The RC 32 can communicate via similar telemetry as the CP 30 to control and/or obtain data from the IPG 10. The patient RC 32 may also be programmable on its own, or may communicate or be linked with the CP 30. The RC 32 may be a dedicated device, including a custom device, a locked off-the-shelf device with specialized software to prevent other uses, or may be a multi-purpose device such as the patient's smart phone.

A charger 36 may be provided to the patient to allow the patient to recharge the IPG 10, if the IPG 10 is rechargeable. In some systems, the IPG 10 is not rechargeable, and so the charger 36 may be omitted. The charger 36 can operate, for example, by generating a varying magnetic field (such as via an inductor) to activate an inductor associated with the IPG 10 to provide power to recharge the IPG battery, using known methods and circuitry.

Some systems may include an external test stimulator (ETS) 38. The ETS 38 can be used to test therapy programs after the lead 12 has been implanted in the patient to determine whether therapy will or can work for the patient 16. For example, an initial implantation of the lead 12 can take place using, for example, a stereotactic guidance system, with the IPG 10 temporarily left out. After a period of healing, the patient may return to the clinic for therapy configuration and testing. The lead 12 may have a proximal end thereof connected to an intermediate connector (sometimes called an operating room cable) that couples to the ETS 38, and the ETS 38 can be programmed using the CP 30 with various therapy programs and stimulation parameters. Once therapy suitability for the patient is established to the satisfaction of the patient 16 and/or physician, the permanent IPG 10 is implanted and the lead 12 is connected thereto, with the ETS 38 then removed from use.

A vagal stimulation system may be provided as shown at 40, located near the vagus nerve. This may be in place of the DBS IPG 10 and lead 12, if desired, or may be an additional stimulator for the patient 16. Stimulation devices may be microstimulators, and may include or exclude a lead, as desired. Some example microstimulators are can be observed in U.S. Pat. No. 8,127,424, the disclosure of which is incorporated herein by reference. Devices, including microstimulators, may be externally powered or internally powered, as desired.

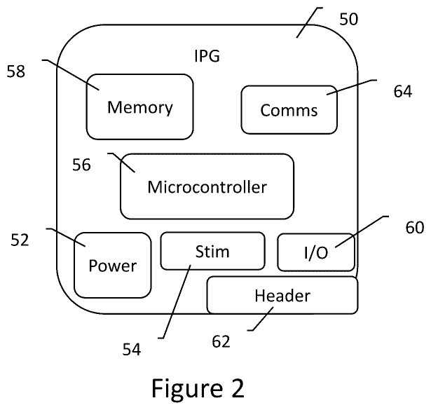

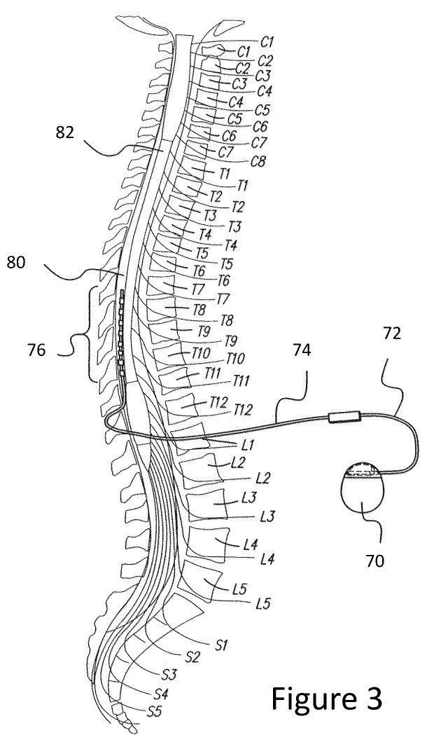

FIG. 2 shows an illustrative IPG in block form. The IPG may have a suitable hermetic housing 50, which may be conductive (titanium, stainless steel, etc.) in order to serve as an additional electrode in the system. Inside housing 50 there is a power supply 52, which may include one or more batteries (rechargeable or not), along with charging circuitry (if rechargeable) and controlled voltage supplies (as desired and suitable to the system). Stimulation circuitry is shown at 54, and provides outputs for the system to use in therapy. A microcontroller 56 is also provided and may be associated with a memory 58. The microcontroller may also be in the form of a microprocessor. Any suitable arrangement of additional systems and circuitry may be included, such as additional logic, communications bus, application specific integrated circuits, etc. The memory 58 may include any of RAM, ROM, and/or Flash memory, or other memory devices/media, and stores machine readable instructions for performing the methods disclosed herein and providing device configurations as described herein. The stimulation circuitry 54 issues therapy pulses, which are directed in accordance with input/output circuitry 60 (which may include a plurality of switches to allow selection of electrodes for use in outputs), that directs signals to and receives signals from a connector block in the header 62. The connector block in the header 62 is part of port for receiving a lead as shown in FIGS. 1 (above) and/or FIG. 3 (below), with individual electrical connectors for each of a plurality of electrodes on the lead(s). Typically, one to four ports are provided, for use with up to four leads, though the present innovation is not limited to a particular lead arrangement.

A communications circuit is also shown at 64. The communications circuit 64 typically includes a resonator, modulator, amplifier and antenna, and may come as a discrete chip. Commercially available chips for Medradio and/or Bluetooth (including Bluetooth Low Energy) can be used, for example. The antenna may be located in the header 62, if desired, to limit signal attenuation due to the housing 50. The communications circuit 64 provides an interface for the device to communicate with external devices including the CP and RC, which may have corresponding circuitry for using the selected communications mode.

The standard approaches to therapy in neuromodulation systems use either current controlled or voltage-controlled therapy generated by a stimulation circuitry 54. The therapy may include, for example, biphasic square waves or monophasic square waves having passive recovery. In general, the amount of current out of an electrode should zero out over time to avoid corrosion at the electrode-tissue interface. For this reason, biphasic pulses, or monophasic pulses with a passive recovery period are typically used. One or several voltage sources may be used, such as with programmable amplifiers or digital to analog conversion circuits that can convert a received therapy command into an analog output voltage, to provide voltage-controlled therapy.

Alternatively, multiple independent current control (MICC) may be used as stimulation circuitry 54. MICC is a stimulus control system that provides a plurality of independently generated output currents that may each have an independent quantity of current. The use of MICC can allow spatially selective fields to be created by therapy outputs. The term “fractionalization” may refer to how the total current issued by the pulse generator via the electrodes is divided up amongst the electrodes of the lead and/or including the pulse generator canister, which can serve as an additional electrode.

Some examples of current or prior versions of IPG circuitry, including in particular the stimulation circuitry 54 but also power 52, I/O 60, and microcontroller 56, as well as planned future examples, may be found in U.S. Pat. No. 10,716,932, the disclosure of which is incorporated herein by reference. Pulse generator circuitry may include that of the various commercially known implantable pulse generators for spinal cord stimulation, Vagus nerve stimulation, and deep brain stimulation as are also well known. Additional examples of circuitry, designs and operation of system devices (IPG, CP, RC, Charger, and ETS, for example) can be found, for example and without limitation, in U.S. Pat. Nos. 6,895,280, 6,181,969, 6,516,227, 6,609,029, 6,609,032, 6,741,892, 7,949,395, 7,244,150, 7,672,734, 7,761,165, 7,974,706, 8,175,710, 8,224,450, and 8,364,278, the disclosures of which are incorporated herein by reference in their entireties.

The circuitry blocks shown in FIG. 2 may be referred to as operational circuitry, and may be described using additional terms specific to particular circuitry functions thereof.

FIG. 3 shows an illustrative spinal cord stimulation SCS system as implanted. In this example, an IPG 70 may be placed near the buttocks or in the abdomen of the patient, with or without a lead extension 72 for coupling to the lead(s) 74 that enter the spinal column. Region 76 at about the level of the lower thoracic or upper lumbar vertebrae may serve as an entry point to the spinal column, where the distal end of the lead 74 with an electrode array may be placed close to the spinal cord 80. Other locations for the IPG 70 and/or lead 74 may be used. For example, sacral nerve stimulation may be performed by positioning the IPG in the lower torso, and extending a lead to near the sacral nerve, as is known in the art, or in the alternative, using a microstimulator. Peripheral nerve stimulation may also be performed, using an IPG and lead positioned at a desired location near the target neural structure, and/or using a microstimulator positioned near the target neural structure. The SCS implementation may include each of the external devices (CP, RC, Charger, ETS) identified in FIG. 1, though not shown in FIG. 2.

There are many potential sources of device problems. These may include for example, lead related issues including lead fracture, lead migration, which can manifest as under-stimulation or over-stimulation, loss of symptom control, and/or intermittent stimulation. Other issues can arise due to hardware failure, system aging, or damage to components if a patient suffers a fall or is exposed, for example, to strong electric fields. Therapy effects can also occur off-target, whether due to changes in the patient's underlying condition or anatomy, including electrode interface changes, or due to movement of leads or electrodes. Rechargeable systems may slowly (or acutely, in the event of premature failure) lose battery capacity and/or capability, and non-rechargeable systems will eventually reach end of life for the battery due to exhaustion of available battery capacity.

Devices have various internal and external ways of monitoring for any change indicative of loss of function or other issues. For example, devices may monitor lead impedance, or may test using electrical field outputs to determine changes in lead position or orientation, and may query the patient using an RC to determine whether the patient has observed changes to therapy, including loss of efficacy. A standard responsive action is to alert the patient and/or physician that a problem has arisen and the patient should schedule an appointment in clinic. This adds to the clinician burden, and leaves patient without desired therapy benefits until the in-clinic appointment takes place. Approaches that will allow a neuromodulation system to adjust therapy either permanently or as a bridge to an in-clinic appointment are desired.

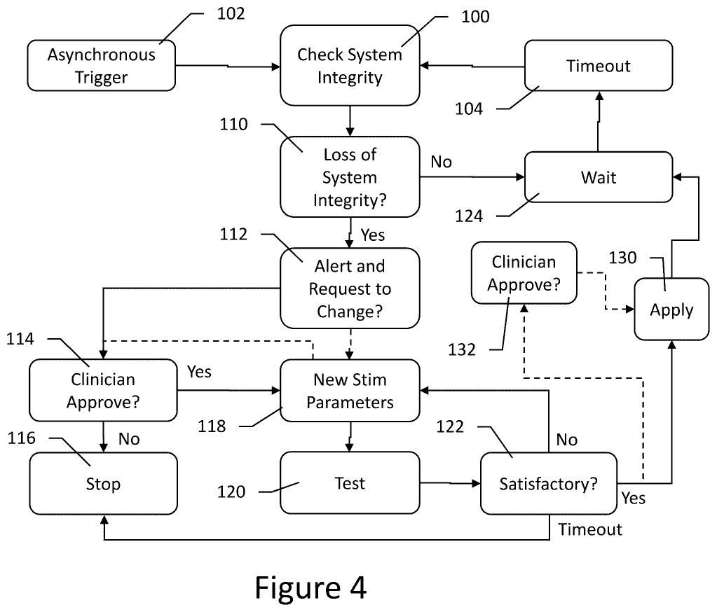

FIG. 4 shows an illustrative method of adapting to loss of system integrity. A system integrity check is performed at block 100. System integrity can be checked in a wide variety of ways. Some illustrative examples include any of the following:

Checking lead integrity through impedance testing. Impedance testing may be performed by issuing a known or controlled amount of current through a given electrode pairing, and sensing a voltage differential. Alternatively, impedance testing may be performed by issuing a known voltage through a given electrode pairing, and sensing a current induced by the voltage. Either way, the simple V=I*R relationship will yield resistance, representing the real part of impedance (the imaginary portion being ignored for this simplified explanation). The current or voltage output can be part of a therapy program, or may be selected specifically for impedance testing. Impedance can then be compared to one or more thresholds. Overly high impedance may indicate any of corrosion of an electrode interface, breaking of a lead conductor, failure in the header/lead port to achieve desired contact between the contact on a lead and a connector in the header/lead port, or dislodgement of the lead from a desired location/position, or other issues. Overly low impedance may indicate shorting due to failure in the lead conductors, fluid ingress to the pulse generator canister or to undesired locations of the header, and/or lead dislodgement, or other issues. Lead dislodgement and lead conductor fracture can be further differentiated by checking impedance on multiple electrodes of a lead; global shifts of the impedances identified may indicate dislodgement, while changes isolated to a single electrode are more likely due to a lead conductor failure, inadequate contact in the header, or damage/corrosion on the electrode interface. Other ways to identify high or low impedance can be used.

Lead migration may also be diagnosed using electrical field measurements. An output of a voltage or current between a first pair of electrodes may be monitored or sensed using other, inactive electrodes. The sensed voltages at the inactive electrodes can be used to determine juxtaposition of multiple leads or electrode arrays, and also to confirm integrity, or identify loss of integrity, on the inactive electrodes, including electrode-tissue interface, conductor integrity in the lead, and contact integrity in the header. For single lead or paddle electrodes, global shifts in impedance may indicate lead migration.

Battery system integrity can be monitored as well. This may be monitored by determining open circuit or lightly loaded voltage of a system battery, which should be expected to decrease over time due to usage and age. The amount of current drawn from the battery may be monitored and the open circuit voltage versus current drawn may compared to a model of normal battery discharge to determine battery fault. An identified battery fault may be characterized as a loss of system integrity. Rechargeable battery behavior during charging may be similarly monitored.

Battery charging system integrity can be monitored as well. For example, temperatures and time required to charge can be monitored during battery recharging to confirm that each of the inductor, rectification circuits, and capacitors in the recharging circuit are performing nominally. Unduly slow charging, or excess heating, may be indicators of charging system fault.

Integrity checking at block 100 may be prompted by an asynchronous trigger 102. For example, identification of a high impedance during therapy delivery can be an asynchronous trigger 102, prompting further impedance testing in block 100. Patient dissatisfaction with therapy can also be an asynchronous trigger 102, and may be indicated by the patient responding to queries from the RC (FIG. 1, at 32) or using an application operated on the RC (such as but not limited to when the RC is a smartphone or other multi-use device). If the RC receives repeated negative responses, the RC may communicate to the IPG a need to check system integrity. Integrity checking at block 100 may also occur at intervals, when a timeout occurs as indicated at 104. For example, integrity may be checked hourly, daily, or weekly (or other longer or shorter interval) may result in a timeout at block 104, prompting integrity check 100, or during specific time periods such as when the patient is deemed to be sleeping due to time of day and/or inactivity as determined using a patient activity or movement monitor.

At block 110, a determination is made as to whether a loss of system integrity has been observed. If not, the system enters a wait state 124, and the method will wait 124 for the next timeout 104 or asynchronous trigger 102 before returning to block 100. If a loss of system integrity has occurred, the method proceeds to block 112.

At block 112, an alert is issued and a request to change therapy is issued. Alerts can be sent to the physician, patient, a manufacturer patient care specialist or patient care center, and/or other caregiver. Block 114 waits for approval by the clinician or other recipient of the alert, who may be remote. For example, an alert to the physician may include the pulse generator communicating to the RC, which can be connected to one or more networks (internet, cellular, etc.) allowing communication directly to the physician, such as by phone call, email or text alert, or communication via a device manufacturer which may facilitate contacting the clinician. After a change is approved, new stimulation parameters are obtained or generated at block 118. If the clinician does not approve of a change, the method stops at 116.

In some examples, the process flows differently as shown by the broken lines.

Here, the loss of system integrity is handled by first obtaining or determining new stimulation parameters at block 118, and transmitting the new stimulation parameters to the physician for approval, so that both the alert and a proposed new therapy parameter set is presented in block 114. In still other examples, the physician approval at 114 is omitted, and the process flows from block 110 to 118 more or less directly. The alert at 112 may also, optionally, include alerting the patient, if desired, such as by issuing tones with the implantable pulse generator, or providing an alert via the patient remote control.

The new stimulation parameters 118 may be generated internally by the IPG, externally by the patient RC, or remotely from a central server or customer relations group managed by a device manufacturer, or remotely from a clinician/physician's office, whether by use of clinician programmer or by use of a computer, tablet or smartphone running an application. In some examples shown below, the new stimulation parameters 118 target a different portion of the neuro-anatomy than the original stimulation.

The new stimulation parameters from block 118 are then tested, as indicated at 120. Testing may be performed in any suitable way. To the extent available, in-patient sensing, such as of evoked response (including but not limited to the evoked compound action potential) may be used to monitor tested therapy efficacy, if desired. For some patients, efficacy may also be measured using tremor monitoring, such as with a motion sensor in or on the patient, whether as part of an implantable system or external, wearable, etc.

The testing at 120 may include, in addition or alternatively, obtaining patient feedback using, for example, the patient RC and/or an application operated on a patient RC. For example, as therapy is tested in block 120, the patient may respond to queries or otherwise input information to the patient RC. For example, the patient may indicate presence or absence of paresthesia, pain, pain relief, tremor, or other sensations or physical or mental experience in relation to the newly tested therapy. A patient may indicate increased sense of agitation or fatigue as inputs as well.

Patient feedback is then used at 122 to determine whether the new stimulation parameters from 118 are satisfactory. “Satisfactory” can mean several different things. In some example, stored or remotely accessible patient data may indicate how the patient initially or previously responded to therapy that had been applied prior to the finding of a loss of system integrity. Comparing results reported by the patient, or measured from the patient (such as by using a motion sensor which may take the form of an accelerometer either located in the IPG or that is part of a wearable device in communication with the IPG or RC, such as a fitness or movement tracker), in response to the new stimulation parameters, to those originally relied upon to make a prior programming decision is one way to determine satisfactory. In another example, the patient may provide their own inputs; which may be as simple as querying the patient via the patient remote control on whether the therapy with new stimulation parameter is working, beneficial, etc. If paresthesia is used the patient may be requested to indicate whether the region (dermatome) in which pathological pain is experienced by the patient is overlapped by the paresthesia.

If the patient experiences tremor that is to be treated, the patient may indicate whether tremor subsides in response to therapy. If an undesired side effect, such as dyskinesia or interrupted sleep, with a previous therapy, absence of the side effect may be confirmed using patient feedback. If data is present in patient records (on the IPG, RC, associated application, connected server, etc.) regarding gait, a gait test may be administered such as via the RC and compared to any of a standard gait assessment, patient's prior performance on such a test, or any other suitable standard/guideline. Gait may also be tested by monitoring motion of the IPG as the patient moves, walks, etc.

If the patient or other feedback at block 122 is not satisfactory according to analysis at block 122, the method may return to block 118 to identify additional stimulation parameters, and the method passes again to blocks 120 and 122. If the process has repeated a preset number of times, a timeout may be declared, and the method stops at 116. In some examples, it may be determined whether additional therapy targets are available in the patient data available to the system and, if not, failure at block 122 to achieve a satisfactory outcome can result in a stop 116.

If the new stimulation parameters are deemed satisfactory at block 122, the method then applies the new stimulation parameters at block 130. Block 130 may, for example, include making the new parameters available as a program that the patient can select and activate on the patient RC. Some examples call for the clinician to approve new therapy parameters at 132, similar to block 114, prior to application at block 130. The method then returns to the wait state at 124 until a next iteration of integrity checking takes place.

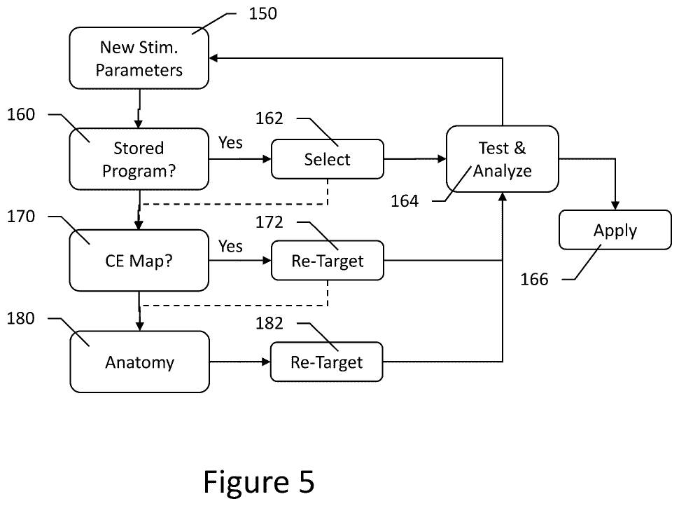

FIG. 5 shows an illustrative method of selecting a new a therapy program. At block 150, a request for new stimulation parameters is made. This may include, for example, reaching block 118 of FIG. 4 in some examples, with or without physician input being requested or obtained. A first source of new stimulation parameters can be a stored program 160. Stored programs 160 may include programs that are stored in the IPG or patient RC, which are not presently in use or which the patient has not been using. Stored programs 160 may also be retrieved remotely, such as from a database of candidate programs that can be remotely queried by the IPG, operating, for example, through the RC to access cellular or internet resources. For example, a physician, clinic, or device manufacturer may have a stored set of candidate therapies, which may be selected from by using any of patient characteristics, lead position, disease state, and/or prior program settings. If available, a stored program is selected at 162. It should be noted that a stored program, to be selected, would need to resolve the underlying issue of loss of integrity. For example, if a “problem” electrode has an impedance out of range, any stored program from block 160 would need to omit the problem electrode from a therapy regimen, such as by assigning no current or voltage to the problem electrode.

It is not expected that block 160 will work very often for most patients, as program configurations at the time of implantation, which are the most likely such source, will not have been designed to avoid using one or more selected electrodes. However, if desired, at implantation or at a programming session, the system and/or physician may determine that a set of back-up programs is to be calculated, where each backup program is designed to account for integrity failures that could cover most situations. For example, given a particular therapy program for a patient that is deemed effective, a set of back-up programs could be automatically generated, each re-creating the central point of stimulation, or stimulation field model of the base program (or doing so with as little change as can be had) while omitting at least one active electrode of the base program.

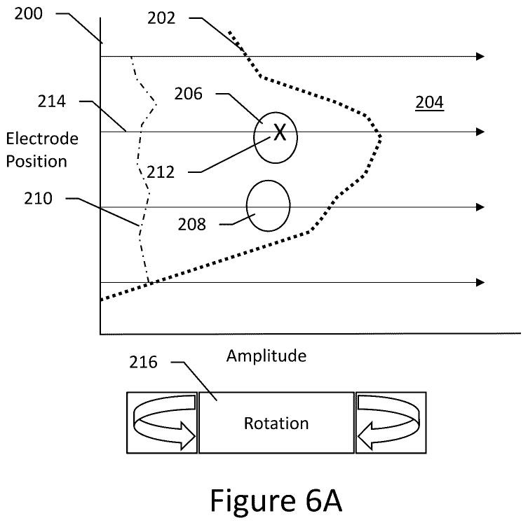

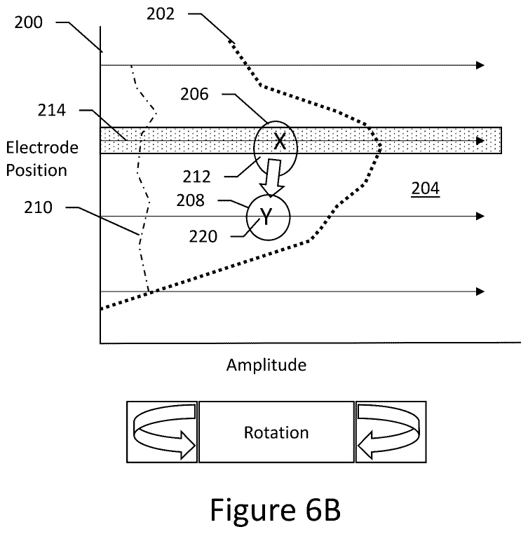

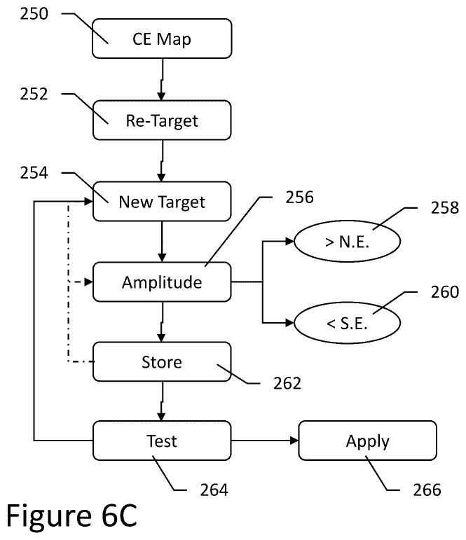

In another example, a clinical effects map (“CE Map”) may be referenced, as indicated at 170. FIGS. 6A-6C, below, illustrate out a CE Map can be used. In particular, a new set of stimulation parameters can be generated using a new CE Map target, at indicated at 172, from the CE Map.

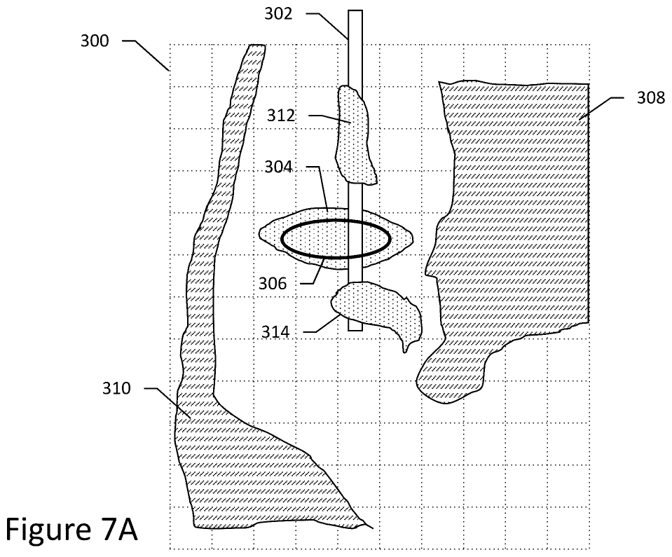

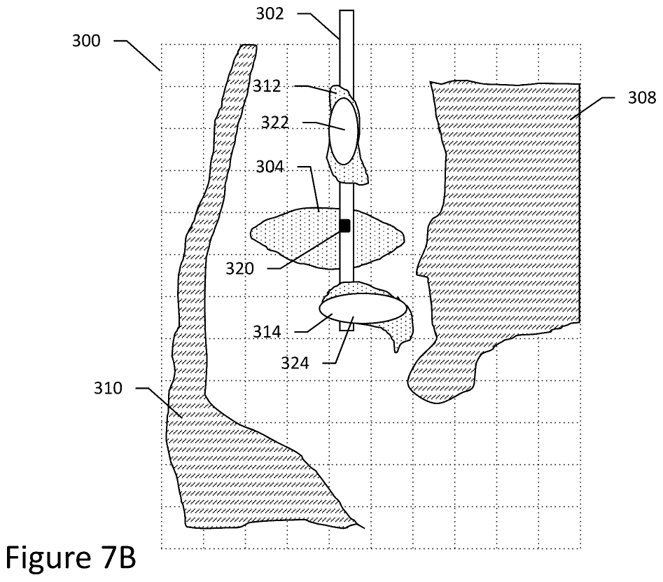

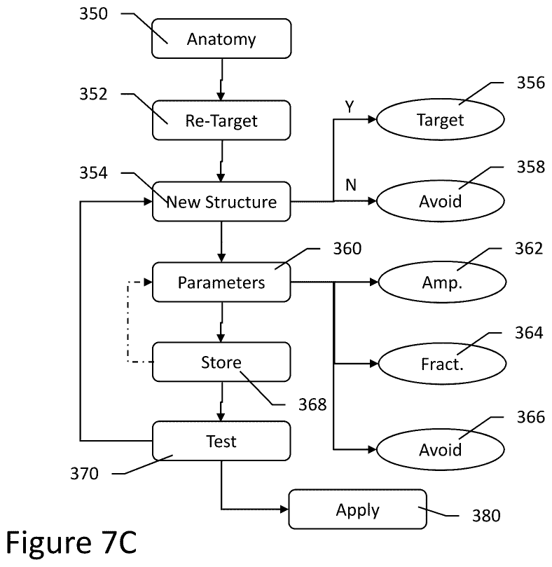

In another example, anatomy data may be referenced, as indicated at 180. FIGS. 7A-7C illustrate methods for using anatomy to generate stimulation parameters. In particular, a new set of stimulation parameters can be generated using a new anatomical target, as indicated at 182.

Whether from block 162, 172, or 182, the new therapy parameters are then tested, and analyzed to see if satisfactory results occur, as indicated at 164. If not, the method reverts to block 150 until or unless a timeout is reached. A timeout may be as discussed previously relative to FIG. 4. Applying the newly selected program at 166 may include, for example, making the new stimulation parameters part of a program that the patient can select and apply using the patient RC, with or without prior approval by a physician.

FIGS. 6A-6C show the use of a clinical effects map (CE Map) to configure a new therapy program. FIG. 6A shows a CE Map 200. The horizontal axis represents the amplitude of stimulus, and the vertical axis represents lead locations, which may be understood as well as the electrode positions on the lead. A side effect line 202 defines the border of side effect region, which shows amplitudes at a given electrode position above which side effects are likely. Line 202 may be determined by testing the patient in clinic, for example. An effectiveness line is also shown at 210, showing amplitude at any given electrode level below which no therapy benefit was observed or is anticipated to occur. The view can be rotated about the lead axis using the selection buttons shown at 216. In the example shown, two “hot spots”, or locations of increased therapy benefit, are highlighted at 206 and 208. A current therapy, marked as the X at 212, is delivered at electrode position 214, and offers a range of amplitudes of increase therapy benefit. FIG. 6B illustrates a response to integrity failure of an electrode located at position 214. With one electrode no longer available, and, in this case, that electrode 214 being level with the hot spot 206, a new therapy target is identified using the CE Map. In this case, therapy is to be targeted to position Y, in the other hot spot region 206. A new set of therapy parameters, centering therapy at Y, indicated at 220, combines therapy amplitudes and electrode positions that can be taken from the CE Map. For example, no current (or voltage) may now be applied to the electrode at position 214, and the stimulation outputs are now generated to position the center of therapy at an electrode position corresponding to location/level Y.

FIG. 6C illustrates a process as in FIGS. 6A-6B in block diagram form. The CE Map is referenced at 250, and retargeting identified at 252. The new target is selected at 254, and amplitude settings are determined at 256. These may include setting the amplitude high enough to be in the targeted sweet spot for the new target, including above the no-effect line (line 210 in FIGS. 6A-6B), as indicated at 258, and below the side effects line (line 202 in FIGS. 6A-6B), as indicated at 260. Data is stored at block 262 and then tested as indicated at 264. If testing is unsatisfactory, the method returns from block 264, and another new target may be identified in block 254. If desired, a plurality of parameter settings directed to the new target may be generated, as indicated by the broken line returns from block 262 to either of 254 or 256, and testing at block 264 may include multiple rounds of testing different amplitudes, current fractionalizations, and/or voltage assignments across multiple electrodes, each of which can be stored in block 262. If testing is a success, the new stimulation parameters are then applied as indicated at 266.

FIGS. 7A-7C show the use of anatomical mapping to configure a new therapy program. FIG. 7A is a visual representation of a mapping 300 of a lead 302 as positioned relative to a target structure 304, showing a representation of a volume of tissue that is stimulated to above an activation threshold, at 306. The tissue activation volume representation at 306 can be called a stimulation field model (SFM), which is used in the art to illustrate how the system is interacting the target structures in the patient. Target structures 304, 312, and 314 may be identified analytically or by a physician as neural structures to which therapy is to be directed to provide therapeutic benefits. Avoid structures shown at 308, 310 may be identified analytically or by a physician as neural structures to avoid stimulating. Analytical identification of neural target or avoid structures may be performed by combining imaging study data (structural MRI, X-Ray, CT Scan, etc.) taken from a patient having lead 302 implanted therein, with databases of anatomy data so that estimates of lead position 302 relative to neural structures can be made. A physician may be asked to confirm or adjust such information.

As shown in FIG. 7B, a problem electrode 320 is identified, for example, as having an out-of-range impedance. In response, the system identifies other potential targets, including region 312 and 314. Possible new therapy volumes are shown at 322, 324, and are calculated based on an aim to deliver stimulus above an activation threshold in the target regions 312, 314. The analysis of SFM, and targeting, may be performed as described in U.S. Pat. No. 11,195,609, the disclosure of which is incorporated herein by reference and describes a process of analytically defining small volumes around the lead and scoring potential therapies based on volumes (target, avoid structure and total volume) that would be activated using a given steering configuration and amplitude.

As between the two available targets 312, 314, a determination may be made as to which one presents a greater risk of stimulating a side effect or avoid structure. In the example shown, region 314 is close to avoid structure 308, and the stimulation field 324 calculated as optimal for region 314 resides very close to the avoid structure 308, and so the other structure 312 may be preferred. Other analyses may be made.

FIG. 7C illustrates a method in block form, using the examples of FIGS. 7A-7B. Here, anatomy data is referenced as indicated at 350. The data may be stored on the IPG, patient RC, or in a remote location accessible by the RC, for example, such as a physician, clinic or manufacturer's server. Retargeting is requested as indicated at 352, in response to an integrity failure. A new target structure 354 is identified in the anatomy data from 350. That the new structure is a target 356, and not an avoid structure 358 are both conditions to meet. Proximity of a new target to an avoid structure may be analyzed; if the new target cannot be stimulated with the given lead position without also stimulating an avoid structure, then the new target may not be considered further, in some examples.

New parameters are then calculated as indicated at 360. The calculation of new parameters may be performed by the IPG or patient RC, or remotely on a physician CP or a remote server operated by a clinic or device manufacturer, as desired. The parameters at 360 include, for example, therapy amplitude 362 and fractionalization 364. The parameters 360 may be determined with consideration of the potential to stimulate avoid structures as indicated at 366. A plurality of test parameters may be calculated for a given new target structure, if desired, as indicated by the broken line arrow from block 368 back to block 360. The resulting new stimulation parameters are then tested at 370, as before. If satisfactory performance cannot be obtained for a given new structure/target, the process may return to block 354 to determine if other structures are available, if desired. If testing is successful and the results are satisfactory, the new parameter set can then be applied as indicated at 380, with or without physician review.

Though not shown in the preceding, a bedside monitor may be used to perform many of the functions of the patient remote. These may include, for example, issuing communication to a remote or central server (such as may be operable by a manufacturer, a clinical practice, or a physician), generating alerts to the patient, and/or calculating any of a new therapy program, metrics for a new therapy program, or changes in patient status that may indicates a loss of system integrity.

Several references in the preceding to activity that a patient RC, a bedside monitor, or an application operated on the patient RC, may be performed as well by an affiliated application operable on any suitable device. For example, the patient may have an RC as well as a smartphone. The smartphone may have thereon an application that can be used to obtain, monitor and/or report out patient feedback. Reporting out patient feedback may use any suitable communications approach, such as by reporting to a remote server via internet communications, or by cellular communications, text messaging, etc., as desired.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein. In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” In the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A method of operation in a neuromodulation system having an implantable pulse generator including a housing containing operational circuitry configured to deliver therapy to the patient using one or more therapy programs, the method comprising:
applying a first set of program parameters to deliver therapy to a patient, the first set of program parameters being in a first therapy program;
checking system integrity;
determining a loss of system integrity;
in response to determining a loss of system integrity, applying a second set of program parameters to deliver therapy to the patient, the second set of program parameters being in a second therapy program;
obtaining patient feedback in response to the application of the second set of program parameters to deliver therapy to the patient; and
determining whether the patient feedback indicates acceptability of the second set of program parameters.

2. The method of claim 1, wherein the neuromodulation system also includes a patient remote control in communication with the implantable pulse generator, wherein:
checking system integrity includes obtaining, from the patient remote control, data indicating whether the patient is satisfied with therapy delivered using the first set of program parameters; and
determining a loss of system integrity includes finding that:
the patient was previously satisfied with therapy delivered using the first set of program parameters; and
that the patient is no longer satisfied with therapy delivered using the first set of program parameters.

3. The method of claim 1, wherein the neuromodulation system includes a patient remote control in communication with the implantable pulse generator, and a lead coupled to the implantable pulse generator, wherein:
checking system integrity includes obtaining one or more of position or impedance data related to the lead, and obtaining, from the patient remote control, data indicating whether the patient is satisfied with therapy delivered using the first set of program parameters; and
determining a loss of system integrity includes finding each of:
the position or impedance data related to the lead has changed from a prior state to a current state; and
the data from the patient remote control indicates:
the patient was previously satisfied with therapy delivered using the first set of program parameters; and
the patient is no longer satisfied with therapy delivered using the first set of program parameters.

4. The method of claim 1, wherein:
the neuromodulation system includes a lead having a proximal end for coupling to the pulse generator and a distal end with a plurality of electrodes thereon, wherein the first therapy program is delivered using a first electrode of the plurality of electrodes on the lead;
the method comprises measuring impedance at the first electrode while therapy is delivered using the first electrode; and
determining a loss of system integrity is performed by finding the measured impedance has crossed above a first threshold or below a second threshold, indicating a potential failure with the first electrode.

5. The method of claim 4, wherein:
the operational circuitry includes a memory storing instructions for executing the one or more therapy programs;
the memory contains a clinical effects map indicating beneficial combinations of therapy amplitude and lead position that cause clinical benefits and side-effect combinations of therapy amplitude and lead position that cause side effects in the patient;
the first therapy program uses a first combination of therapy amplitude and lead position that cause clinical benefits having a first location on the clinical effects map; and
the method comprises determining the second set of program parameters in response to the loss of system integrity by identifying a second location on the clinical effects map that uses a combination of therapy amplitude and lead position which the clinical effects map indicates will cause clinical benefits and not side effects.

6. The method of claim 4, wherein:
the operational circuitry includes a memory storing instructions for executing the one or more therapy programs;
the memory contains an anatomical mapping of target locations and avoid locations, relative to the lead in the patient;
the first therapy program is configured to issue therapy to a first target location; and
the method comprises determining the second set of program parameters in response to the loss of system integrity by identifying a second target location that is not the first target location, and configuring the second set of program parameters to issue therapy to the second target location.

7. The method of claim 4, wherein:
the neuromodulation system includes a patient remote control having a memory storing a clinical effects map indicating benefit combinations of therapy amplitude and lead position that cause clinical benefits and side-effect combinations of therapy amplitude and lead position that cause side effects in the patient;
the first therapy program uses a first combination of therapy amplitude and lead position that cause clinical benefits having a first location on the clinical effects map;
the method includes:
identifying a second location on the clinical effects map that uses a second combination of therapy amplitude and lead position which the clinical effects map indicates will cause clinical benefits and not side effects;
determining the second set of program parameters from the second combination of therapy amplitude and lead position; and
the patient remote control communicating the second set of program parameters to the implantable pulse generator.

8. The method of claim 4, wherein:
the neuromodulation system includes a patient remote control having a memory storing an anatomical mapping of target locations and avoid locations, relative to the lead in the patient;
the first therapy program is configured to issue therapy to a first target location; and
the method includes:
determining the second set of program parameters in response to the loss of system integrity by identifying a second target location that is not the first target location, and configuring the second set of program parameters to issue therapy to the second target location; and
the patient remote control communicating the second set of program parameters to the implantable pulse generator.

9. The method of claim 4, wherein:
the operational circuitry includes a memory storing instructions for executing at least the first set of program parameters and the second set of program parameters, and an active list identifying which of the plurality of sets of program parameters are available to be selected by a patient for use at any given time;
the method includes determining that the second set of program parameters is not in-use at the time the loss of system integrity occurs, and the first set of program parameters is in use at the time the loss of system integrity occurs; and
the method includes removing the first set of program parameters from the active list, and adding the second set of program parameters to the active list.

10. The method of claim 4, wherein the second set of program parameters does not use the first electrode in therapy delivery.

11. The method of claim 1, further comprising:
generating an alert to request a change in response to the loss of system integrity; and
receiving a response to the alert, approving the change;
wherein the method includes waiting to apply the second set of program parameters to deliver therapy to the patient until after receiving the response approving the change.

12. The method of claim 11, wherein the step of generating an alert to request a change includes sending the second set of program parameters with the request.

13. The method of claim 11, wherein the step of generating an alert to request a change includes alerting a physician.

14. The method of claim 1, wherein the system also includes a patient remote control configured to present queries to a patient and receive responses therefrom, wherein the step of obtaining patient feedback includes communicating to the patient remote control to present a query to the patient, and then receiving an indication of the patient feedback from the patient remote control.

15. The method of claim 1, wherein the step of obtaining patient feedback includes monitoring a patient motion sensor to analyze one or more of gait or tremor.

16. The method of claim 15, wherein the patient motion sensor is contained in or on the implantable pulse generator.

17. The method of claim 15, wherein the patient motion sensor is a component of a wearable device, and the implantable pulse generator contains communication circuitry configured to communicate with the wearable device.

18. The method of claim 15, wherein:
the system also includes a patient remote control adapted to communicate with the implantable pulse generator and having an interface for communicating with the patient;
the patient motion sensor is a component of a wearable device; and
the patient remote control is further adapted to communicate with the wearable device.

19. A neuromodulation system comprising an implantable pulse generator including a housing containing operational circuitry having therein a microcontroller and a memory, the memory storing patient data and readable instructions for one or more programs for treating the patient; wherein the operational circuitry is configured to:
apply a first set of program parameters to deliver therapy to a patient, the first set of program parameters stored in the memory;
check system integrity;
determine a loss of system integrity;
obtain or determine a second set of program parameters in response to the loss of system integrity;
apply the second set of program parameters to deliver therapy to the patient;
obtain patient feedback in response to the application of the second set of program parameters to deliver therapy to the patient; and
determine whether the patient feedback indicates acceptability of the second set of program parameters.

20. The neuromodulation system of claim 19, further comprising a patient remote control, wherein the operational circuitry comprises a communications circuit adapted to communicate with the patient remote control, wherein:
the operational circuitry is configured to check system integrity by obtaining, from the patient remote control, data indicating whether the patient is satisfied with therapy delivered using the first set of program parameters; and
the operational circuitry determines a loss of system integrity in response to the data from the patient remote control indicating:
the patient was previously satisfied with therapy delivered using the first set of program parameters; and
that the patient is no longer satisfied with therapy delivered using the first set of program parameters.
